(19) United States
(12) Patent Application Publication
(10) Pub. No.: US 2022/0370496 A1
(43) Pub. Date: Nov. 24, 2022

(54) HER3 PULSED DC1 THERAPY

(71) Applicant: H. LEE MOFFITT CANCER CENTER AND RESEARCH INSTITUTE, INC., Tampa, FL (US)

(72) Inventors: Brian J. CZERNIECKI, Tampa, FL (US); Krithika N. Kodumudi, Tampa, FL (US)

(21) Appl. No.: 17/642,809

(22) PCT Filed: Sep. 14, 2020
(86) PCT No.: PCT/US2020/050689

§ 371 Date: Mar. 14, 2022

Related U.S. Application Data
(60) Provisional application No. 62/900,107, filed on Sep. 13, 2019.

(51) Int. Cl. A61K 35/17; A61K 31/337; A61P 35/00; G01N 33/68

(52) U.S. Cl. CPC A61K 35/17; A61K 31/337; A61P 35/00; G01N 33/6878; G01N 33/6866; G01N 2333/57

(57) ABSTRACT

Disclosed are compositions and methods comprising the administration of pulsed dendritic cells and an immunoregulator molecule inhibitor for the treatment of cancer.

This application claims the benefit of U.S. Provisional Application No. 62/900,107, which is incorporated herein by reference in its entirety.

### I. BACKGROUND

The aggressive features of various cancer types are mainly driven by oncodrivers such as HER2, HER3, EGFR, c-MET that are critically involved in cell growth, proliferation, survival and differentiation. Overexpression of these oncodrivers has associated with poor prognosis and a key player in tumor cell resistance to targeted therapies. Additionally, oncodriver expression, such as HER3 overexpression in triple negative breast cancer (TNBC), has been shown to be involved in tumor progression. Previous attempts using peptide vaccines to boost CD8 immune responses have shown some efficacy but have not generated complete tumor regression. One possibility is that CD8s function sub-optimally without CD4s. What are needed are new peptide-based immunotherapies that account for both CD8 and CD4 T cells.

### II. SUMMARY

Disclosed are methods and compositions related to novel combination therapies comprising oncodriver pulsed dendritic cells and methods of identifying oncodriver epitopes for pulsing dendritic cells.

In one aspect, disclosed herein are anti-cancer therapies comprising at least one dendritic cell pulsed with at least one HER-3 CD4+ T cell epitope such as, for example, an epitope from the extracellular domain (ECD) of HER-3 (such as, for example, SEQ ID NO: 1, SEQ ID NO: 2, SEQ ID NO: 3, SEQ ID NO: 4, SEQ ID NO 8, and/or SEQ ID NO: 9) or intracellular domain (ICD) of HER-3 (such as, for example, SEQ ID NO: 5, SEQ ID NO: 6, SEQ ID NO: 7, SEQ ID NO: 10, and/or SEQ ID NO: 11) and/or at least one HER-2 CD4+ T cell epitope (such as, for example, SEQ ID NO: 12, SEQ ID NO: 13, SEQ ID NO: 14, SEQ ID NO: 15, SEQ ID NO: 16, and/or SEQ ID NO: 17). Thus, in one aspect, the dendritic cell can be pulsed with SEQ ID NO: 1, SEQ ID NO: 2, SEQ ID NO: 3, SEQ ID NO: 4, SEQ ID NO: 5, SEQ ID NO: 6, SEQ ID NO: 7. SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and/or SEQ ID NO: 17.

Also disclosed herein are anti-cancer therapies of any preceding aspect, further comprising at least one anti-cancer agent (such as, for example Taxol).

In aspect, disclosed herein are anti-cancer therapies of any of preceding aspect, wherein the HER-3 CD4+ T cell epitope pulsed dendritic cell and/or HER-2 CD4+ T cell epitope pulsed dendritic cell is activated with IL-12 prior to administration.

Also disclosed herein are methods of treating, preventing, inhibiting, or reducing a cancer or metastasis in a subject comprising administering the anti-cancer therapy of any preceding aspect. In one aspect, the least one anti-cancer agent is administered systemically and/or the at least one HER-3 CD4+ T cell epitope pulsed dendritic cell and/or HER-2 CD4+ T cell epitope pulsed dendritic cell is administered intratumorally.

In one aspect, disclosed herein are methods of treating, preventing, inhibiting, reducing, ameliorating, and/or preventing a cancer and/or metastasis in a subject comprising administering to the subject an HER-3 CD4+ T cell epitope (such as, for example, an epitope from the extracellular domain (ECD) of HER-3 (such as, for example, SEQ ID NO: 1, SEQ ID NO: 2, SEQ ID NO: 3, SEQ ID NO: 4, SEQ ID NO 8, and/or SEQ ID NO: 9) or intracellular domain (ICD) of HER-3 (such as, for example, SEQ ID NO: 5, SEQ ID NO: 6, SEQ ID NO: 7, SEQ ID NO: 10, and/or SEQ ID NO: 11) and/or at least one HER-2 CD4+ T cell epitope (such as, for example, SEQ ID NO: 12, SEQ ID NO: 13, SEQ ID NO: 14, SEQ ID NO: 15, SEQ ID NO: 16, and/or SEQ ID NO: 17).

Also disclosed herein are methods of treating, preventing, inhibiting, reducing, ameliorating, and/or preventing a cancer and/or metastasis of any preceding aspect, wherein the method further comprises administering to the subject at least one anti-cancer agent (such as, for example, Taxol).

In one aspect, disclosed herein are methods of treating, preventing, inhibiting, reducing, ameliorating, and/or preventing a cancer and/or metastasis of any preceding aspect, wherein the at least one dendritic cell is removed from the subject and pulsed with the HER-3 CD4+ T cell epitope and/or a HER-2 CD4+ T cell epitope ex vivo.

Also disclosed herein are methods of treating, preventing, inhibiting, reducing, ameliorating, and/or preventing a cancer and/or metastasis of any preceding aspect, wherein the at least one pulsed dendritic cell is administered intratumorally and/or the at least one anti-cancer agent is administered systemically.

In one aspect, disclosed herein are methods of treating, preventing, inhibiting, reducing, ameliorating, and/or preventing a cancer and/or metastasis of any preceding aspect, wherein the at least one pulsed dendritic cell is administered at least 1, 2, 3, 4, 5, 6, 7, 8, 9, 10, 11, 12, 18, 24, 30, 36 hours, 2, 3, 4, 5, 6, 7, 8, 9, 10, 11, 12, 13, 14, 21, 28, 30, 31, 45 days, 2, 3, 4, 5, or 6 months prior to administration of the at least one anti-cancer agent; wherein the at least one pulsed dendritic cell is administered concurrently with the at least anti-cancer agent; or, wherein the at least one anti-cancer agent is administered at least 1, 2, 3, 4, 5, 6, 7, 8, 9, 10, 11, 12, 18, 24, 30, 36 hours, 2, 3, 4, 5, 6, 7, 8, 9, 10, 11, 12, 13, 14, 21, 28, 30, 31, 45 days, 2, 3, 4, 5, or 6 months prior to administration of the pulsed dendritic cells.

In one aspect, disclosed herein are methods of identifying class-II promiscuous epitopes (i.e., a promiscuous CD4+ T cell epitope) comprising creating an overlapping peptide fragments of an oncodriver (including, but not limited to HER1, HER2, HER3, EGFR, c-MET, BRAF, KIT, AR, ER, KRAS, TP53, and/or APC), pulsing autologous dendritic cells from normal donors and cancer patients (such as, for example TNBC patients), co-culturing said pulsed dendritic cells with T cells for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days, and measuring the IFN-γ production following re-stimulation with iDCs.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows a schematic representation of the screening of Class-II promiscuous peptides for vaccine development.

FIG. 2 shows a schematic representation of peptide library screening and identification of immunogenic peptide candidates. Red cells represent positive peptide pools and blue cells represent negative peptide pools.

FIGS. 3A and 3B show the identification of immunogenic CD4+ HER3 epitopes from intracellular and extracellular HER3 library using sequential screening. FIG. 3A shows a Bar graph represents the extracellular domain (ECD) screening of individual peptides. FIG. 3B shows a bar graph representing the intracellular domain (ICD) screening of individual peptides.

FIGS. 4A and 4B show sequential screenings of the HER3 ECD and ICD revealed three immunogenic HER3 ICD epitopes (4A) and four immunogenic HER3 ECD epitopes (4B).

FIG. 5 shows that restimulation of splenocytes from HER3-DC1 vaccinated mice with HER3 peptides had significant increase in IFN-γ production.

FIG. 6 shows that all six HER3 peptides generate an immune response in vaccinated naïve mice.

FIGS. 7A and 7B show that HER3-DC1 vaccination delays tumor growth in the preventive setting of TNBC model. FIG. 7A shows the delay in tumor growth. FIG. 7B shows the survival of vaccinated mice relative to controls.

FIG. 8 shows the treatment schedule of mouse experiments.

FIGS. 9A and 9B show that therapeutic vaccination with HER3-DC1 delays tumor Growth and improves survival in MT901 when administered after a tumor has been established. FIG. 9A shows the delay in tumor growth. FIG. 9B shows the survival of vaccinated mice relative to controls.

FIG. 10 shows the therapeutic efficacy of intratumoral HER3-DC1 and Taxol. Taxol alone group received two injections of taxol. DC alone group received DCs once weekly beginning on day 5. DC+Taxol group received two injections of taxol before beginning DC treatment. One mouse in the DC+Taxol group reached endpoint and died before functional analysis.

FIG. 11 shows HER2-DC1 versus Her2/Her3-DC1 targeted immunotherapy for BC-LMD murine model

FIG. 12 shows the results for HER2-DC1 versus Her2/Her3-DC1 targeted immunotherapy in TUBO-LMD Re-challenge experiments.

FIG. 13 shows a first LMD challenge and treatment using Her2/Her3-DC1 targeted immunotherapy in comparison with a cancer re-challenge TUB O-LMD using Her2/Her3-DC1 targeted immunotherapy.

FIG. 14 shows the HER3-DC vaccination and preventive effect on tumor growth in 4T1 model. Balb/c mice were vaccinated with either HER3 ECD peptide-pulsed DC or HER3 ECD+ICD peptide-pulsed DC vaccines (10⁶ cells/mouse), administered subcutaneously twice/week for a total of six doses. Mice were challenged subcutaneously with 4T1 tumor cells (5×10⁴ cells/mouse) in the flank. Tumor growth was monitored every 2-3 days until endpoint was reached.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims that follow, reference will be made to a number of terms which shall be defined to have the following meanings:

“Optional” or “optionally” means that the subsequently described event or circumstance may or may not occur, and that the description includes instances where said event or circumstance occurs and instances where it does not.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc.

A “binding molecule” or “antigen binding molecule” (e.g., an antibody or antigen-binding fragment thereof) as provided herein refers in its broadest sense to a molecule that specifically binds an antigenic determinant. In one embodiment, the binding molecule specifically binds to an immunoregulator molecule (such as for example, a transmembrane SEMA4D (CD100) polypeptide of about 150 kDa or a soluble SEMA4D polypeptide of about 120 kDa). In another embodiment, a binding molecule is an antibody or an antigen binding fragment thereof, e.g., MAb 67 or pepinemab.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular immunoregulator molecule inhibitor or oncodriver pulsed dendritic cell is disclosed and discussed and a number of modifications that can be made to a number of molecules including the immunoregulator molecule inhibitor or oncodriver pulsed dendritic cell are discussed, specifically contemplated is each and every combination and permutation of immunoregulator molecule inhibitor or oncodriver pulsed dendritic cell and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Patients presenting with DCIS in general have excellent prognosis however those presenting at age <40, African American females, and ER″g DCIS have modestly increased risk of dying of subsequent BC that neither surgery nor radiation appears to prevent. These patients are typically either treated with mastectomy because of the size of area of calcifications or treated with strong neoadjuvant chemotherapy regimens of carboplatin, taxotere with trastuzumab and pertuzumab (PTCH) or taxol and trastuzumab (TH) with good survival but to suffer the long term consequences of extensive surgery, radiation and chemotherapy. In a day of personalized medicine these patients need more personalized effective therapy that both reduces the odds of subsequent BC mortality and at the same time reduces the overtreatment they receive from chemotherapy, a year of trastuzumab, radiation and often mastectomies. Patients with metastatic breast cancer (MBC) are in desperate need of new immunotherapies to reduce mortality especially those that become resistant to targeted therapies.

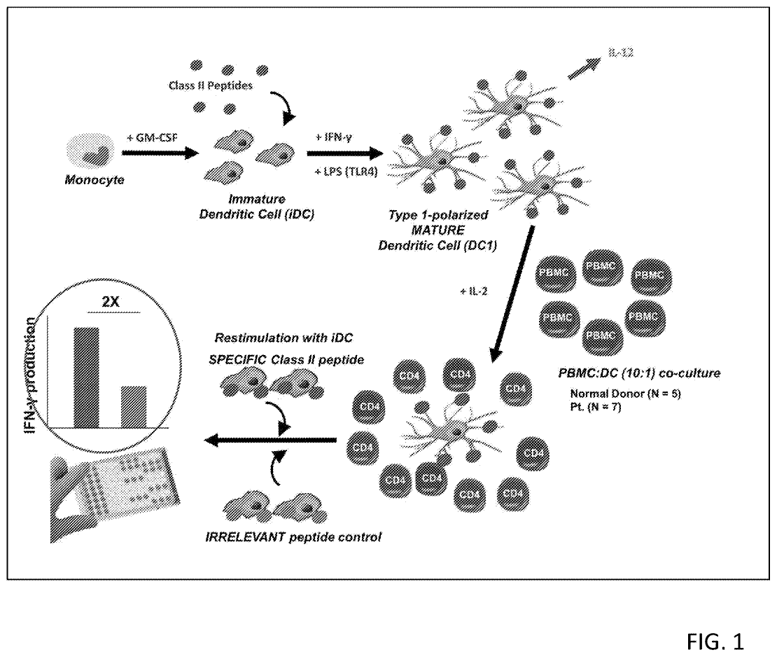

Oncodriver expression, such as HER3 overexpression in triple negative breast cancer (TNBC), has been shown to be involved in tumor progression. Previous attempts using peptide vaccines to boost CD8 immune responses have shown some efficacy but have not generated complete tumor regression. One possibility is that CD8s function sub-optimally without CD4s. Interestingly, there is a loss of the anti-HER3 CD4+Th1 immune response during breast tumorigenesis. Using the extracellular domain (ECD) of HER3 as a candidate “oncodriver” tumor antigen, immunogenic HER3 peptides that demonstrate class II promiscuity and generate anti-HER3 CD4 immunity were investigated for inclusion in dendritic cell (DC) vaccine development (FIG. 1). Accordingly, in one aspect, disclosed herein are anti-cancer therapies comprising at least one dendritic cell pulsed with at least one HER-3 CD4+ T cell epitope.

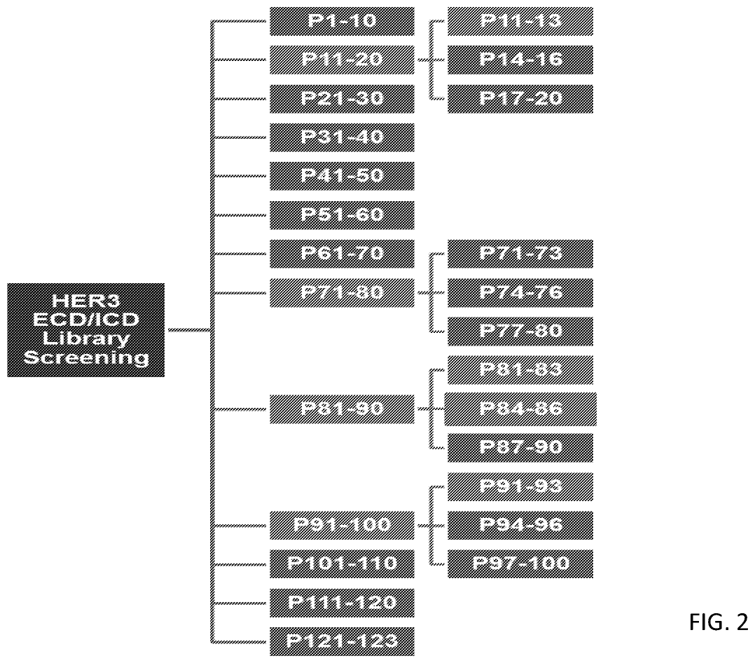

Peptide libraries of overlapping 15 amino-acid long peptide fragments were generated from the HER3 ECD and ICD (see FIG. 2 and Table 1) and from HER-2 (Table 2). Thus, it is understood and herein contemplated that the HER3 CD4+ T cell epitope can be generated from the ECD (such as, for example, SEQ ID NO: 1, SEQ ID NO: 2, SEQ ID NO: 3, SEQ ID NO: 4, SEQ ID NO: 8, or SEQ ID NO: 9) or the ICD (such as, for example, SEQ ID NO: 5, SEQ ID NO: 6, SEQ ID NO: 7, SEQ ID NO: 10, or SEQ ID NO: 11) of HER3. In one aspect, the HER2+ T cell epitope can be SEQ ID NO: 12, SEQ ID NO: 13, SEQ ID NO: 14, SEQ ID NO: 15, SEQ ID NO: 16, or SEQ ID NO: 17.

TABLE 1
HER3 CD4+ T cell epitopes

| | | Homology with |
|---|---|---|
| HER3 Epitopes Identified | Amino Acid Sequences | Mouse HER3 |
| HER3 ECD P12 (aa 57-71) | EVVMGNLEIVLTGHN (SEQ ID NO: 1) | 100% |
| HER3 ECD P81 (aa 401-415) | SWPPHMHNFSVFSNL (SEQ ID NO: 2) | 100% |
| HER3 ECD P84 (aa 416-430) | TTIGGRSLYNRGFSL (SEQ ID NO: 3) | 100% |
| HER3 ECD P91 (aa 450-464) | SAGRIYISANRQLCY (SEQ ID NO: 4) | 87% |
| HER3 ICD P38 (aa 850-864) | VADFGVADLLPPDDK (SEQ ID NO: 5) | 100% |
| HER3 ICD P41 (aa 865-879) | QLLYSEAKTPIKWMA (SEQ ID NO: 6) | 93% |
| HER3 ICD P52 (aa 920-934) | VPDLLEKGERLAQPQ (SEQ ID NO: 7) | 93% |
| HER ECD P12 (aa 56-70) | CEVVMGNLEIVLTGH (SEQ ID NO: 8) | |
| HER3 ECD P91 (aa 451-465) | AGRIYISANRQLCYH (SEQ ID NO: 9) | |
| HER3 ICD P86 (aa 1090-1114) | GCLASESSEGHVTGS (SEQ ID NO: 10) | |
| HER3 ICD P89 (aa 1115-1129) | EAELQEKVSMCRSRS (SEQ ID NO: 11) | |

TABLE 2
Her2 CD4+ T cell epitopes

| HER2 Epitopes | |
|---|---|
| Identified | Acid Sequences Amino |
| HER2 P42-56 | HLDMLRHLYQGCQVV (SEQ ID NO: 12) |
| HER2 P98-114 | RLRIVRGTQLFEDNYAL |
| | (SEQ ID NO: 13) |
| HER2 P328-345 | TQRCEKCSKPCARVCYGL |
| | (SEQ ID NO: 14) |
| HER2 P776-790 | GVGSPYVSRLLGICL (SEQ ID NO: 15) |
| HER2 P927-941 | PAREIPDLLEKGERL (SEQ ID NO: 16) |
| HER2 P1166-1180 | TLERPKTLSPGKNGV (SEQ ID NO: 17) |

It is understood and herein contemplated that in some instances the addition of an anti-cancer agent can be beneficial to the ultimate treatment goals of a patient. Accordingly, the disclosed anti-cancer therapies and methods of treating, inhibiting, reducing, and/or preventing a cancer using said anti-cancer therapies can further comprise one or more anti-cancer agents. It is also understood and herein contemplated that more than one population of pulsed dendritic cells with each population of pulsed dendritic cells being pulsed with the same or different oncodrivers can be used. Thus, in one aspect, disclosed herein are anti-cancer therapies comprising i) one or more HER-3 CD4+ T cell epitope pulsed dendritic cells and/or one or more HER-2 CD4+ T cell epitope pulsed dendritic cell and ii) one or more anti-cancer agents.

As noted above, it is intended herein that the disclosed methods of treating, inhibiting, reducing, and/or preventing cancer can augmented with any therapeutic treatment of a cancer including, but not limited surgical, radiological, and/or pharmaceutical treatments of a cancer. As used herein, “surgical treatment” refers to tumor resection of the tumor by any means known in the art. Similarly, “pharmaceutical treatment” refers to the administration of any anti-cancer agent known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil-Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil-Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil-Topical), Fluorouracil Injection, Fluorouracil-Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil-Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), rintatolimod, celecoxib, Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Also contemplated herein are chemotherapeutics that are PD1/PDL1 blockade inhibitors (such as, for example, lambrolizumab, nivolumab, pembrolizumab, pidilizumab, BMS-936559, Atezolizumab, Durvalumab, or Avelumab). Thus, disclosed herein are anti-cancer therapies comprising i) a HER-3 CD4+ T cell epitope pulsed dendritic cell and/or a HER-2 CD4+ T cell epitope pulsed dendritic cell and ii) one or more anti-cancer agents, wherein the at least one anti-cancer agent comprises Taxol.

In one aspect, it is understood and herein contemplated that the pulsed dendritic cells can be activated prior to administration as well as prior to being pulsed with the oncodriver. Activation of the dendritic cells (DC1) can be achieved by contacting the cells with IFN-γ, TNFα, CD40, IL21, and/or IL-12. Accordingly, disclosed herein are anti-cancer therapies or methods of treating, preventing, reducing, and/or inhibiting a cancer or metastasis, wherein the HER-3 CD4+ T cell epitope pulsed dendritic cell and/or HER-2 CD4+ T cell epitope pulsed dendritic cell is activated with IL-12 prior to administration.

In one aspect, it is further understood that the subject's own dendritic cells can be removed and pulsed ex vivo and transferred back to the subject for use in the disclosed anti-cancer combination therapies for treating, preventing, reducing, and/or inhibiting a cancer. Thus, disclosed herein are methods of treating, preventing, inhibiting, or reducing a cancer or metastasis, wherein the at least one dendritic cell is removed from the subject and pulsed with the HER-3 CD4+ T cell epitope ex vivo.

It is understood and herein contemplated that the disclosed anti-cancer therapies can be used to treat, prevent, reduce, and/or inhibit any disease where uncontrolled cellular proliferation occurs such as cancers including primary and distant tumors. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumors, myelomas, AIDS-related lymphomas or sarcomas, metastatic cancers, or cancers in general.

A representative but non-limiting list of cancers that the disclosed compositions can be used to treat, prevent, reduce, and/or inhibited is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer (including triple negative breast cancer (TNBC), metastatic breast cancer (MBC), ductal carcinoma in situ (DCIS), and invasive breast cancer (IBC)), and epithelial cancer, renal cancer, genitourinary cancer, pulmonary cancer, esophageal carcinoma, head and neck carcinoma, large bowel cancer, hematopoietic cancers; testicular cancer; colorectal cancer, prostatic cancer, or pancreatic cancer. Thus, in one aspect, disclosed herein are methods of treating, preventing, reducing, and/or inhibiting a cancer (such as, for example, breast cancer (including triple negative breast cancer, metastatic breast cancer (MBC), ductal carcinoma in situ (DCIS), and invasive breast cancer (IBC)), melanoma, colorectal cancer, pancreatic cancer, and prostate cancer and including primary and distant tumors) in a subject comprising administering the anti-cancer combination therapy of any preceding aspect. For example, disclosed herein are methods of treating, preventing, inhibiting, or reducing a cancer or metastasis in a subject comprising administering to the subject an HER-3 CD4+ T cell epitope (such as, for example, an epitope from the extracellular domain (ECD) of HER-3 (such as, for example, SEQ ID NO: 1, SEQ ID NO: 2, SEQ ID NO: 3, SEQ ID NO: 4, SEQ ID NO 8, and/or SEQ ID NO: 9) or intracellular domain (ICD) of HER-3 (such as, for example, SEQ ID NO: 5, SEQ ID NO: 6, SEQ ID NO: 7, SEQ ID NO: 10, and/or SEQ ID NO: 11) and/or at least one HER-2 CD4+ T cell epitope (such as, for example, SEQ ID NO: 12, SEQ ID NO: 13, SEQ ID NO: 14, SEQ ID NO: 15, SEQ ID NO: 16, and/or SEQ ID NO: 17). Thus, in one aspect, the dendritic cell can be pulsed with SEQ ID NO: 1, SEQ ID NO: 2, SEQ ID NO: 3, SEQ ID NO: 4, SEQ ID NO: 5, SEQ ID NO: 6, SEQ ID NO: 7. SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and/or SEQ ID NO: 17.

It is understood and herein contemplated that the disclosed anti-cancer combination therapies can be administered via any route determined to be appropriate by the attending physician. Administration” to a subject includes any route of introducing or delivering to a subject an agent either locally and/or systemically. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intratumoral, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another. In one aspect, disclosed herein are anti-cancer therapies and methods treating, preventing, reducing, and/or inhibiting a cancer of any preceding aspect; wherein the at least one anti-cancer agent is administered systemically and/or the oncodriver pulsed dendritic cell (such as, for example a HERS CD4+ T cell epitope (such as SEQ ID NOs: 1-11) pulsed DC or a HER2 CD4+ T cell epitope (such as SEQ ID NOs: 12-17) pulsed DC) is administered intratumorally.

It is understood and herein contemplated that while a single administration of the components of the disclosed anti-cancer combination therapies (i.e., the pulsed dendritic cells and/or the immunoregulator molecule inhibitor) would be ideal, not every patient will respond in the same manner Thus, in one aspect, disclosed herein are anti-cancer combination therapies methods treating, preventing, reducing, and/or inhibiting a cancer; wherein the at least one pulsed dendritic cell is administered at least 1, 2, 3, 4, 5, 6, 7, 8, 9, 10, 11, 12, 13, 14, 15, 16, 17, 18, 19, 20, 21, 22, 23, or 24 times per day or at least 1, 2, 3, 4, 5, 6, 7, 8, 9, 10, 11, 12, 13, or 14 times per week for at least 1, 2, 3, 4, 5, 6, 7, 8, 9, 10, 11, 12, 13, 14 days, 3, 4, 5, 6, 7, 8, 9, 10, 11, 12 weeks. Also disclosed herein are anti-cancer combination therapies methods treating, preventing, reducing, and/or inhibiting a cancer of any preceding aspect; wherein the at least one anti-cancer agent is administered at least 1, 2, 3, 4, 5, 6, 7, 8, 9, 10, 11, 12, 13, 14, 15, 16, 17, 18, 19, 20, 21, 22, 23, or 24 times per day or at least 1, 2, 3, 4, 5, 6, 7, 8, 9, 10, 11, 12, 13, or 14 times per week for at least 1, 2, 3, 4, 5, 6, 7, 8, 9, 10, 11, 12, 13, 14 days, 3, 4, 5, 6, 7, 8, 9, 10, 11, 12 weeks. It is further understood and herein contemplated that the order and duration of the administered components can vary as appropriate for the subject being treated. In one aspect, disclosed herein are anti-cancer combination therapies methods treating, preventing, reducing, and/or inhibiting a cancer; wherein the pulsed dendritic cells are administered at least 1, 2, 3, 4, 5, 6, 7, 8, 9, 10, 11, 12, 18, 24, 30, 36 hours, 2, 3, 4, 5, 6, 7, 8, 9, 10, 11, 12, 13, 14, 21, 28, 30, 31, 45 days, 2, 3, 4, 5, or 6 months prior to administration of the at least one anti-cancer agent; are administered concurrently with the at least one anti-cancer agent; or wherein the at least one anti-cancer agent is administered at least 1, 2, 3, 4, 5, 6, 7, 8, 9, 10, 11, 12, 18, 24, 30, 36 hours, 2, 3, 4, 5, 6, 7, 8, 9, 10, 11, 12, 13, 14, 21, 28, 30, 31, 45 days, 2, 3, 4, 5, or 6 months prior to administration of the pulsed dendritic cells.

Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 ug/kg to up to 100 mg/kg of body weight or more per day, depending on the factors mentioned above.

### C. METHODS OF IDENTIFYING CLASS-II PROMISCUOUS EPITOPES

These results represent a novel strategy to rapidly and reproducibly identify class II-promiscuous epitopes from any tumor antigen using a DC1-Th1 platform. Therefore, this approach can also be used to target other oncodrivers such as human epidermal growth factor receptor (HER) 1 (HER1), HER2, HER3, epidermal growth factor receptor (EGFR), c-Mesenchymal to Epithelial Transition (MET), B-Rapidly Accelerated Fibrosarcoma (BRAF), KIT, Androgen Receptor (AR), Estrogren Receptor (ER), Kirsten rat sarcoma (KRAS), TP53, or APC. In one aspect, disclosed herein are methods of identifying class-II promiscuous epitopes comprising creating an overlapping peptide fragments of an oncodriver (including, but not limited to HER1, HER2, HER3, EGFR, c-MET, BRAF, KIT, AR, ER, KRAS, TP53, and/or APC), pulsing autologous dendritic cells from normal donors and cancer patients (such as, for example TNBC patients), co-culturing said pulsed dendritic cells with T cells for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days, and measuring the IFN-γ production following re-stimulation with iDCs.

This approach can also be utilized to generate antigen-specific CD4 T cells for the development of adoptive cell therapy (ACT).

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Identification and Efficacy of a Novel DC1-Th1 Platform

Most breast tumors express oncodrivers including HER2, EGFR (ER+ and TNBC), C-MET (TNBC) and HER3 (ER+, HER2, and TNBC). These oncodrivers or downstream pathways are often targeted by many therapeutics and patients with metastatic breast cancer (MBC) are often treated with targeted agents such as anti-estrogens, CDK4/6 inhibitors, HER2 directed therapies and AKT inhibitors. Most of these patients, however, become resistant to therapies or stop responding and progress. Thus, these patients are in need of additional therapies. Checkpoint therapies have shown promising but limited effectiveness in MBC so identifying effective new immunotherapies that may be combined with targeted agents to make them more effective in MBC would be highly desirable. Oncodrivers may be critical appropriate targets of the immune response as indicated by the data below.

Healthy adult women possess unusually high, pre-existing Th1 immunity against HER2 and HER3 in the peripheral blood. These TbetPc's Th1 cells, however, are progressively lost during tumorigenesis. This deficit is first detectable at the ductal carcinoma in situ (DCIS) stage and becomes profoundly suppressed by stage I invasive breast cancer (IBC) stage. Previous attempts using peptide vaccines to boost CD8 immune responses have shown some efficacy but have not generated complete tumor regression. One possibility is that CD8s function sub-optimally without CD4s. Interestingly, there is a loss of the anti-HER3 CD4+Th1 immune response during breast tumorigenesis. Using the extracellular domain (ECD) of HER3 as a candidate “oncodriver” tumor antigen, immunogenic HER3 peptides that demonstrate class II promiscuity and generate anti-HER3 CD4 immunity were investigated for inclusion in dendritic cell (DC) vaccine development

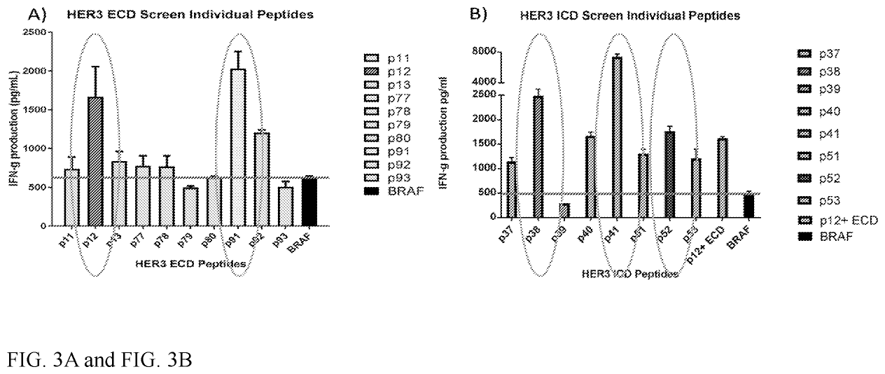

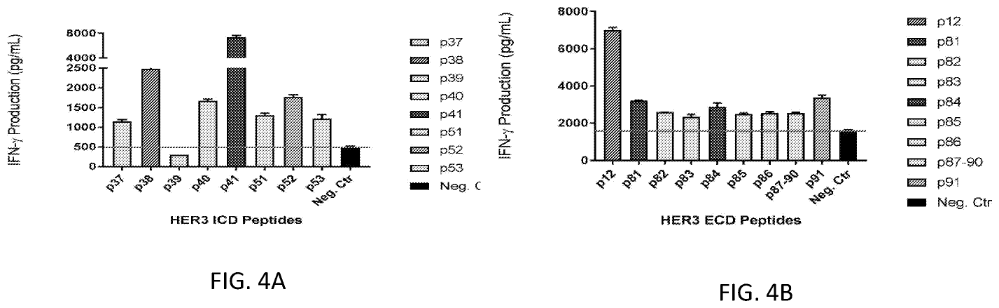

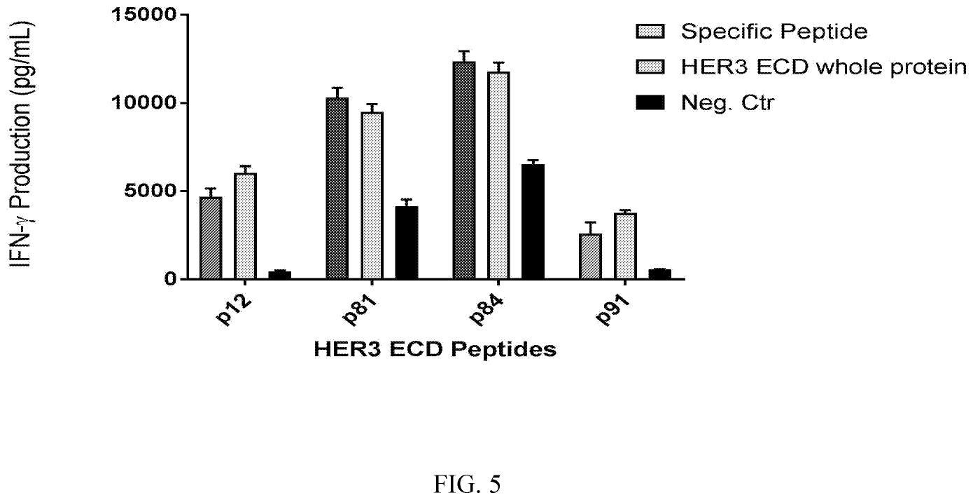

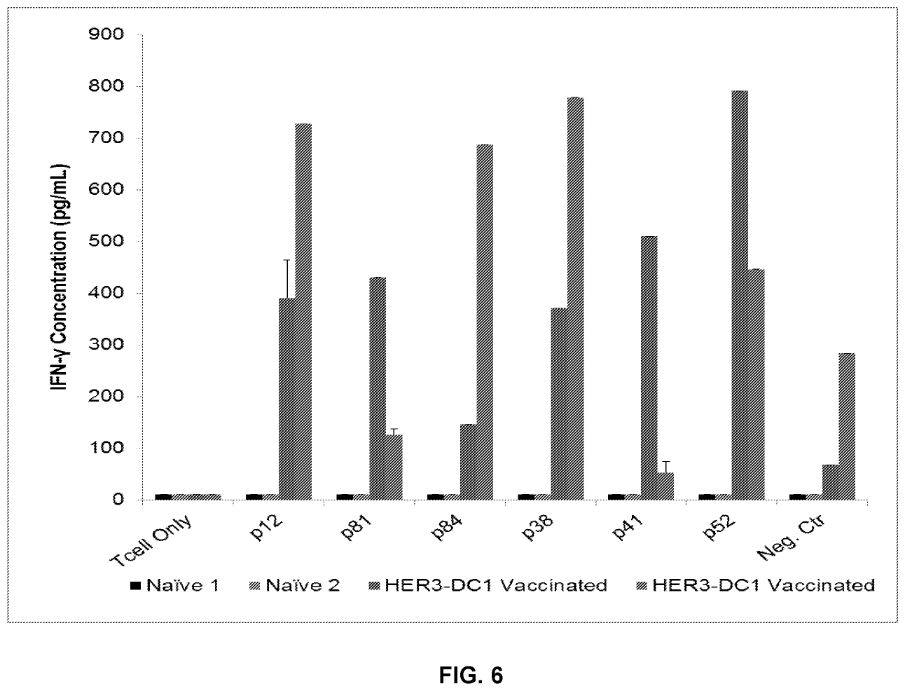

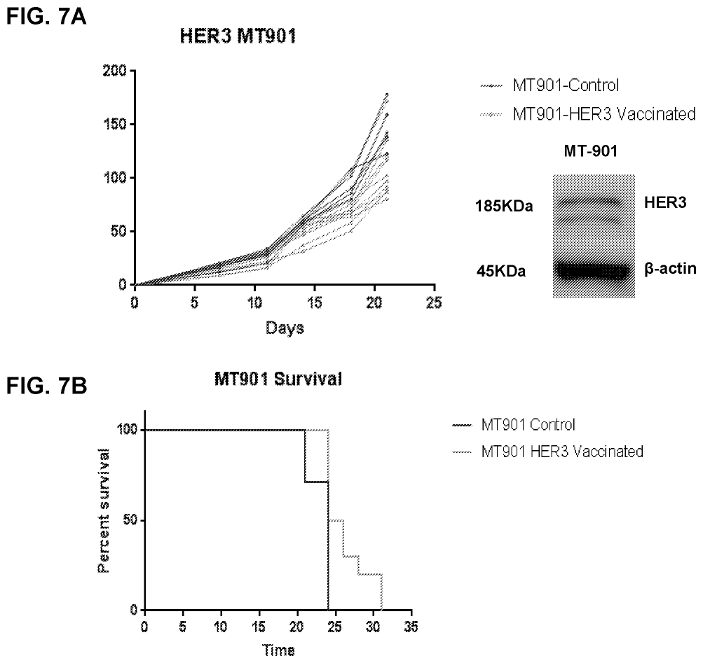

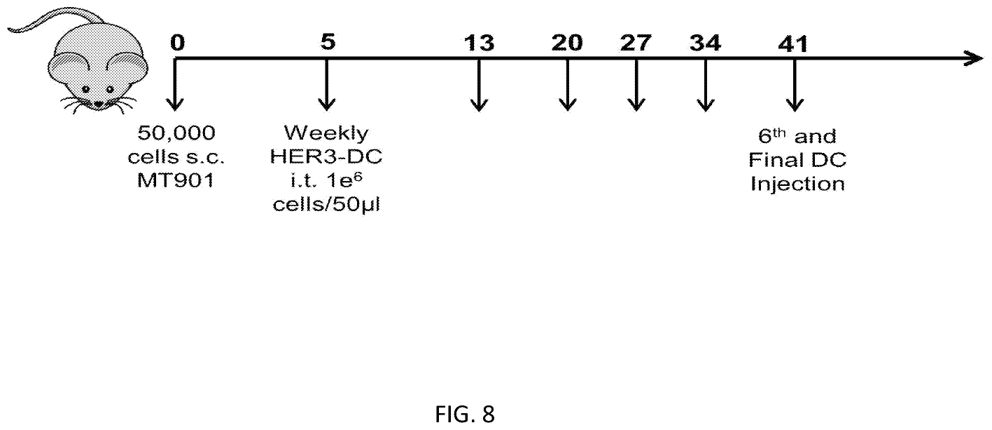

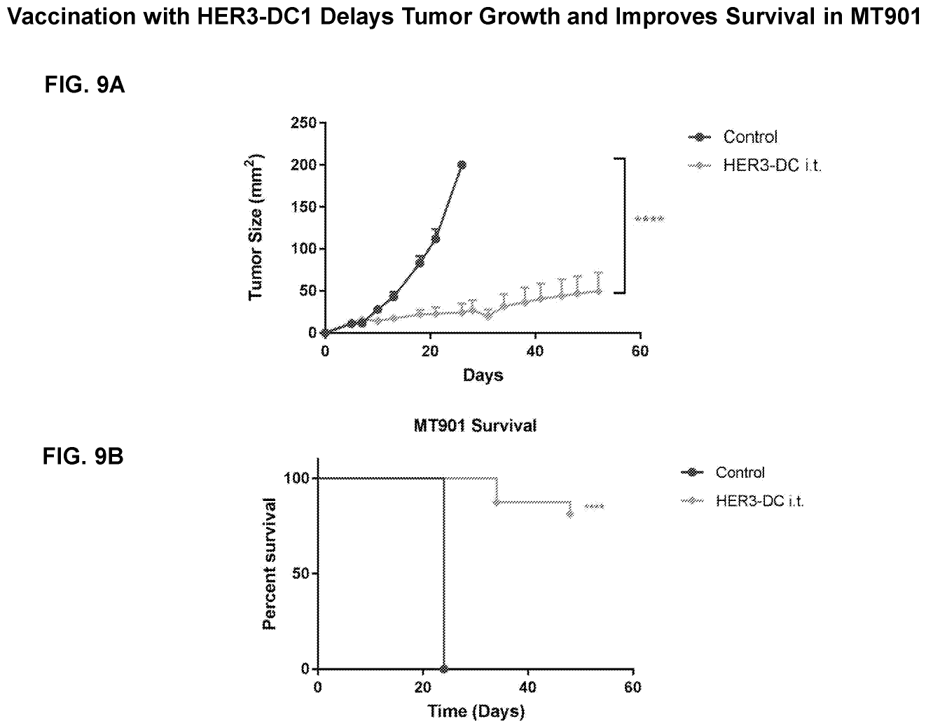

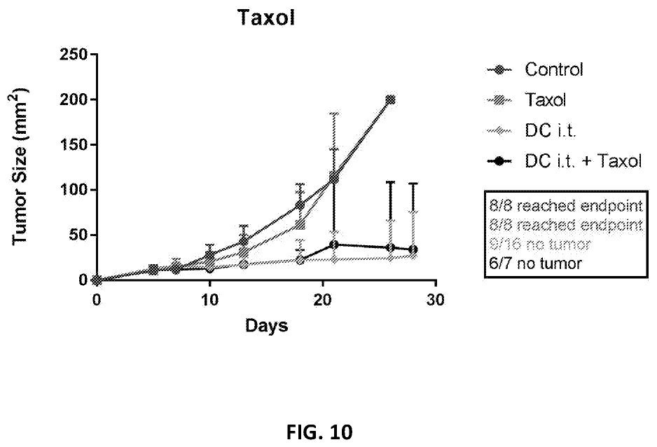

Peptide libraries of overlapping 15 amino-acid long peptide fragments were generated from the HER3 ECD and ICD (see Table 1). Autologous monocyte-derived DCs from normal donors and TNBC patients were pulsed with HER3 peptides (HER3-DC), matured to type I DCs (DC1) and co-cultured with T cells for one week. Antigen specificity was measured by IFN-γ production after re-stimulation with iDCs (see FIGS. 3A, 3B, 4A, and 4B). Since there is 90-100% homology between human and mouse HER3, ECD and ICD pulsed DC1s were used to generate an immune response in a Balb/c mouse model (see FIG. 5). MT901, a mouse TNBC cell line, was injected in Balb/c mice and treatment began once tumors were palpable. HER3-DC1 vaccine was injected intratumorally once a week for six weeks. HER3 immune responses were measured in spleens, lymph nodes, and tumors post-vaccination. Three immunogenic HER3 ICD epitopes and four immunogenic HER3 ECD epitopes were reproducibly identified across seven TNBC patients and five normal donors (FIGS. 4A and 4B). The HER3 epitopes identified demonstrate class II promiscuity across HLA-DR, DP, and DQ. All samples demonstrated successful sensitization to the individual peptides as well as to the native ECD or ICD (FIG. 6). Vaccination with HER3-DC1 in Balb/c mice delayed tumor growth and generated an anti-HER3 an immune response in challenge experiments to determine if vaccination can prevent, inhibit, and/or reduce the onset of cancer (FIGS. 7A and 7B). This data indicates that successful peptide candidates were identified for a HER3 DC vaccine. To investigate how the peptides would perform in the context of a therapeutic treatment of an established cancer, 5×10⁵ MT901 cells were delivered to a Balb/c mouse s.c. and 5 days later weekly administration of 1×10⁶ HER3 pulsed DC cells began and continued for six weeks (FIG. 8). HER3-pulsed DC1 significantly delayed tumor growth and improved survival (FIGS. 9A and 9B). To determine if the therapeutic or preventive effect could be improved in combination with an anti-cancer agent, mice were administered HER3-pulsed DC1 in combination with Taxol. 30 days after tumor cells were transferred to the mice, control mice and mice receiving taxol alone had established tumors. By contrast, mice receiving either HER3-pulsed DC1 or a combination of HER3-pulsed DC1 and taxol had no established tumors (FIG. 10).

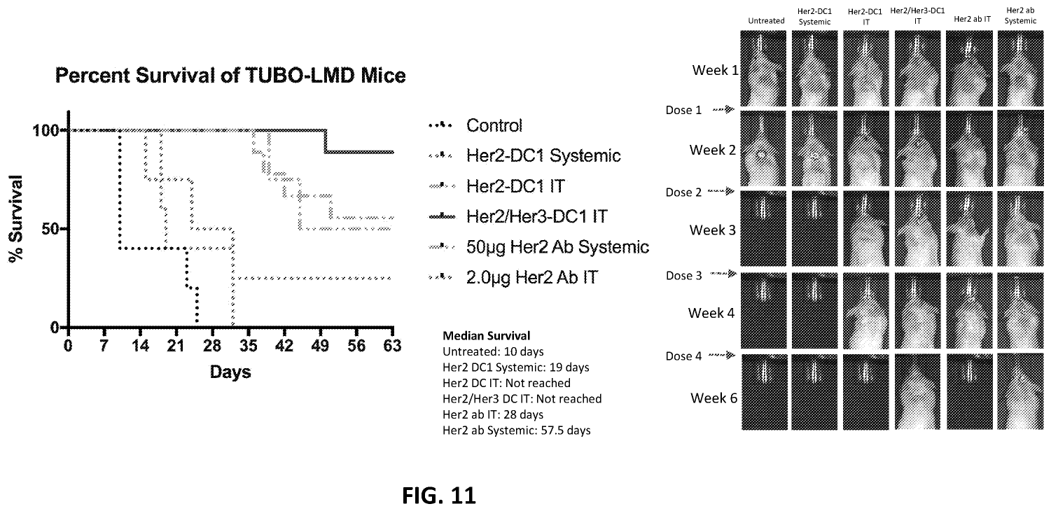

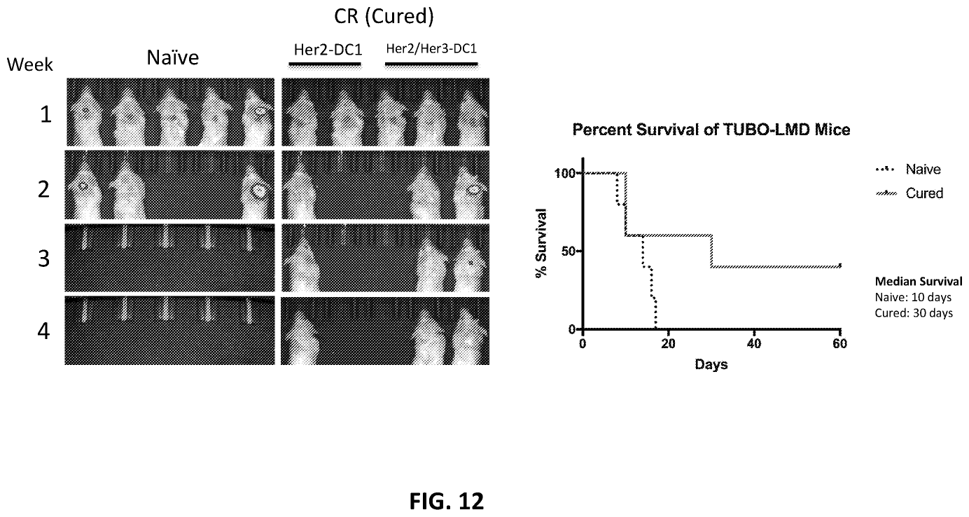

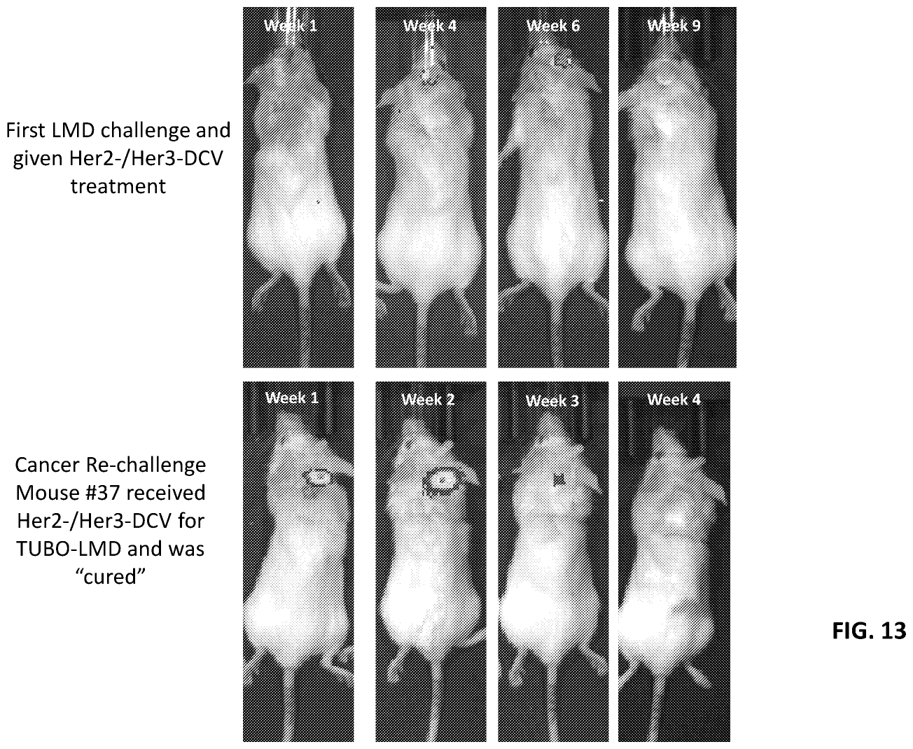

Following this success, the BC-LMD murine model was utilized to test the efficacy of the Her2/Her3-DC1 targeted immunotherapy during first challenge and re-challenge (FIGS. 11, 12, and 13). Her2-DC1 IT and Her2/Her3-DC1 IT groups did significantly better than other treatment arms (excluding Her2 ab Systemic group) (FIG. 11). However, some of the Her2-DC1 IT mice developed LMD much later (week 5 and on). Nevertheless, most of the Her2/Her3-DC1 IT mice are still “healthy” (no signs of physical weaknesses) or LMD detection. As observed in FIG. 11, Her2/Her3-DC1 IT group had the best overall survival. Looking at TUB O-LMD re-challenge data, 66% of the Her2/Her3-DC1 IT group survived the re-challenge in comparison to only 33% of the Her2-DC1 IT mice. In fact, as observed in FIG. 13, mice receiving the Her2/Her3-DC1 IT were able to successfully ablate the tumor to below detectable levels.

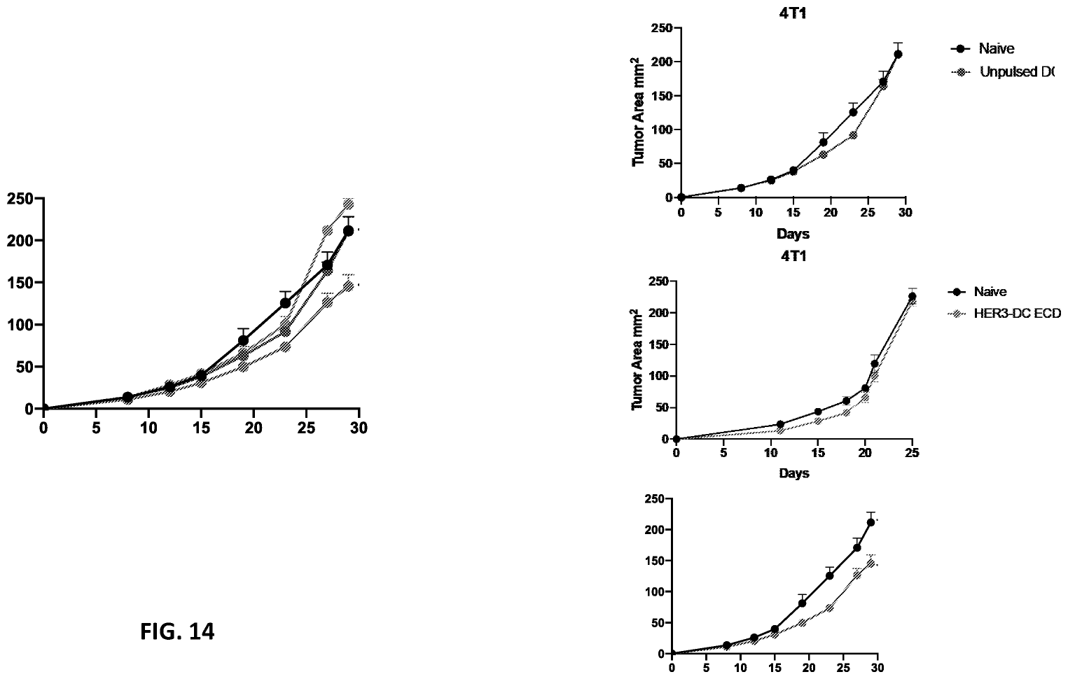

Using the triple negative breast cancer (TNBC) 4T1 model, the prevention effect of the HER-3-Dc vaccination was measured (FIG. 14). Compared to naïve control, significant delay in tumor growth was observed in mice receiving ECD+ICD peptide-pulsed DC vaccine. However, mice receiving HER3 ECD-pulsed DC had tumor growth comparable to naïve control group. Therefore, both ECD and ICD peptides are required for optimal preventive effect of the HER3-DC vaccine.

### 2. Example 2: Phase IIa Study of Alpha-DC1 Vaccines Against HER2/HER3, Chemokine Modulation (CKM) Regimen and Pembrolizumab in Patients with Asymptomatic Brain Metastasis from Triple Negative Breast Cancer (TNBC) or HER2⁺ Breast Cancer (HER2±BC)

Brain metastases develop in up to 50% patients with metastatic triple negative breast cancer (TNBC) and HER2₊ BC and are an increasing source of morbidity and mortality. HERS, overexpressed in Her2⁺ brain-metastatic breast cancer (BMBC) and TNBC, is a resistance factor to HER2-targeted therapies and a driver of CNS metastasis. Progression of HER2+BC and TNBC is associated with loss of anti-HER2 and anti-HERS immunity. We have demonstrated that αDC1 loaded with glioma-specific peptides induce intratumoral production of chemokines (CXCL9, CXCL10, CXCL11, CCLS) which attract CXCR3- and CCRS-expressing cytotoxic T-lymphocytes (CTLs) and T-helper 1 (Th1) cells to brain tumors, inducing clinical responses and long-term disease stabilization in patients with aggressive recurrent primary brain tumors. Our preclinical data show that Chemokine modulating (CKM) regimen [rintatolimod, interferon (IFN)-α2b and COX-2 inhibitor] also selectively attracts effector CTLs and Th1 cells (but not suppressive Tregs or MDSCs) into tumors. Importantly, CKM preferentially activates tumor—rather than healthy tissues, providing rationale for its systemic use to promote local CTL accumulation in tumor lesions. We hypothesize that anti-HER2/3 type 1 polarized DC1s in combination with CKM and anti-PD1 will result in in improved Th1/CTL response against HER2/3 epitopes, reduce brain recurrence and systemic progression.

This phase II single-arm, non-randomized multicenter study (NCT04348747). Eligibility includes patients ≥18 years, ECOG PS ≤1, normal marrow and organ function with TNBC and Her2+ BMBC who receive αDC1 q2 weeks×3, with CKM [200 mg IV rintatolimod, IFN-α20 million units/m²IV, celecoxib 200 mg oral BID] on days 1-3 with second and third dose of αDC1, followed by pembrolizumab 200 mg IV. Thereafter, pembrolizumab is given every 3 weeks; along with αDC1 and CKM every 3 months as booster dose until disease progression, intolerable side effects or withdrawal from study for up to 24 months. Baseline and 3-week post-CKM treatment biopsies are required for six patients. Primary objective is CNS response rate (RR) by RANO-BM criteria. Secondary objectives include non-CNS RR per RECIST v1.1, median CNS, non-CNS and overall progression-free survival (PFS), median overall survival (OS) and safety.

SEQUENCES

SEQ ID NO: 1 HER3 ECD P12 (aa 57-71)
EVVMGNLEIVLTGHN
SEQ ID NO: 2 HER3 ECD P81 (aa 401-415)
SWPPHMHNFSVFSNL
SEQ ID NO: 3 HER3 ECD P84 (aa 416-430)
TTIGGRSLYNRGFSL
SEQ ID NO: 4 HER3 ECD P91 (aa 450-464)
SAGRIYISANRQLCY
SEQ ID NO: 5 HER3 ICD P38 (aa 850-864)
VADFGVADLLPPDDK
SEQ ID NO: 6 HER3 ICD P41 (aa 865-879)
QLLYSEAKTPIKWMA
SEQ ID NO: 7 HER3 ICD P52 (aa 920-934)
VPDLLEKGERLAQPQ
SEQ ID NO: 8 HER3 ECD P12 (aa 56-70)
CEVVMGNLEIVLTGH
SEQ ID NO: 9 HER3 ECD P91 (aa 450-465)
SAGRIYISANRQLCYH
SEQ ID NO: 10 HER3 ICD P86 (aa 1090-1114)
GCLASESSEGHVTGS
SEQ ID NO: 11 HER3 ICD P89 (aa 1115-1129)
EAELQEKVSMCRSRS
SEQ ID NO: 12 HER2 MHC Class II P42-56
HLDMLRHLYQGCQVV
SEQ ID NO: 13 HER2 MHC Class II P98-114
RLRIVRGTQLFEDNYAL
SEQ ID NO: 14 HER2 MHC Class II P328-345
TQRCEKCSKPCARVCYGL
SEQ ID NO: 15 HER2 MHC Class II P776-790
GVGSPYVSRLLGICL
SEQ ID NO: 16 HER2 MHC Class II P927-941
PAREIPDLLEKGERL
SEQ ID NO: 17 HER2 MHC Class II P1166-1180
TLERPKTLSPGKNGV
SEQ ID NO: 18 HLA A2 restricted HER2 peptide
(cross reacts with HER3) P369-377
KIFGSLAFL
SEQ ID NO: 19 HLA A2 restricted HER2 peptide
(cross reacts with HER3) P698-697
RLLQETELV

1. An anti-cancer therapy comprising at least one dendritic cell pulsed with at least one HER-3 CD4+ T cell epitope and/or at least one HER-2 CD4+ T cell epitope.

2. The anti-cancer therapy of claim 1, wherein the HER-3 CD4+ T cell epitope is located in the extracellular domain of HER3.

3. The anti-cancer therapy of claim 2, wherein the HER-3 CD4+ T cell epitope comprises SEQ ID NO: 1, SEQ ID NO: 2, SEQ ID NO: 3, SEQ ID NO: 4, SEQ ID NO: 8, and/or SEQ IDNO: 9.

4. The anti-cancer therapy of claim 1, wherein the HER-3 CD4+ T cell epitope is located in the intracellular domain of HER3.

5. The anti-cancer therapy of claim 4, wherein the HER-3 CD4+ T cell epitope comprises SEQ ID NO: 5, SEQ ID NO: 6, SEQ ID NO: 7, SEQ ID NO: 10, and/or SEQ ID NO: 11.

6. The anti-cancer therapy of claim 1, wherein the HER-2 CD4+ T cell epitope comprises SEQ ID NO: 12, SEQ ID NO: 13, SEQ ID NO: 14, SEQ ID NO: 15, SEQ ID NO: 16, and/or SEQ ID NO: 17.

7. The anti-cancer therapy of claim 1, further comprising at least one anti-cancer agent.

8. The anti-cancer therapy of claim 6, wherein in the at least one anti-cancer agent comprises Taxol.

9. The anti-cancer therapy of claim 1, wherein the HER-3 CD4+ T cell epitope pulsed dendritic cell and/or HER-2 CD4+ T cell epitope pulsed dendritic cell is activated with IL-12 prior to administration.

10. A method of treating a cancer in a subject comprising administering the anti-cancer therapy of claim 1.

11. The method of treating a cancer of claim 10, wherein the at least one anti-cancer agent is administered systemically.

12. The method of treating a cancer of claim 10, wherein the at least one HER-3 CD4+ T cell epitope pulsed dendritic cell and/or HER-2 CD4+ T cell epitope pulsed dendritic cell is administered intratumorally.

13. The method of treating a cancer of claim 10, wherein the cancer is a breast cancer.

14. The method of treating a cancer of claim 13, wherein the breast cancer comprises triple negative breast cancer.

15-22. (canceled)

23. The method of treating a cancer of claim 10, wherein the at least one dendritic cell is removed from the subject and pulsed with the HER-3 CD4+ T cell epitope and/or the Her-2 CD4+ T cell epitope ex vivo.

24-27. (canceled)

28. The method of treating a cancer of claim 10, wherein the at least one pulsed dendritic cell is administered at least 1, 2, 3, 4, 5, 6, 7, 8, 9, 10, 11, 12, 18, 24, 30, 36 hours, 2, 3, 4, 5, 6, 7, 8, 9, 10, 11, 12, 13, 14, 21, 28, 30, 31, 45 days, 2, 3, 4, 5, or 6 months prior to administration of the at least one anti-cancer agent.

29. The method of treating a cancer of claim 10, wherein the at least one pulsed dendritic cell is administered concurrently with the at least anti-cancer agent.

30. The method of treating a cancer of claim 10, wherein the at least one anti-cancer agent is administered at least 1, 2, 3, 4, 5, 6, 7, 8, 9, 10, 11, 12, 18, 24, 30, 36 hours, 2, 3, 4, 5, 6, 7, 8, 9, 10, 11, 12, 13, 14, 21, 28, 30, 31, 45 days, 2, 3, 4, 5, or 6 months prior to administration of the pulsed dendritic cells.

31. A method of identifying a CD4+ T cell epitope of an oncodriver useful in the pulsing of dendritic cells, comprising
a. creating an overlapping peptide fragments of an oncodriver;
b. pulsing autologous dendritic cells from normal donors and cancer patients (such as, for example TNBC patients);
c. co-culturing said pulsed dendritic cells with T cells; and
d. measuring the IFN-γ production following re-stimulation with iDCs.

32. The method of claim 31, wherein the oncodriver comprises human epidermal growth factor receptor (HER) 1 (HER1), HER2, HER3, epidermal growth factor receptor (EGFR), c-Mesenchymal to Epithelial Transition (MET), B-Rapidly Accelerated Fibrosarcoma (BRAF), KIT, Androgen Receptor (AR), Estrogren Receptor (ER), Kirsten rat sarcoma (KRAS), TP53, or APC.

33. The method of claim 31, wherein the pulsed dendritic cells are co-cultured with T cells for at least 7 days.


SEQUENCE LISTING (19 sequences)

SEQ ID NO: 1 - protein, 15 aa, Artificial Sequence
  Synthetic construct
EVVMGNLEIVLTGHN

SEQ ID NO: 2 - protein, 15 aa, Artificial Sequence
  Synthetic construct
SWPPHMHNFSVFSNL

SEQ ID NO: 3 - protein, 15 aa, Artificial Sequence
  Synthetic construct
TTIGGRSLYNRGFSL

SEQ ID NO: 4 - protein, 15 aa, Artificial Sequence
  Synthetic construct
SAGRIYISANRQLCY

SEQ ID NO: 5 - protein, 15 aa, Artificial Sequence
  Synthetic construct
VADFGVADLLPPDDK

SEQ ID NO: 6 - protein, 15 aa, Artificial Sequence
  Synthetic construct
QLLYSEAKTPIKWMA

SEQ ID NO: 7 - protein, 15 aa, Artificial Sequence
  Synthetic construct
VPDLLEKGERLAQPQ

SEQ ID NO: 8 - protein, 15 aa, Artificial Sequence
  Synthetic construct
CEVVMGNLEIVLTGH

SEQ ID NO: 9 - protein, 16 aa, Artificial Sequence
  Synthetic construct
SAGRIYISANRQLCYH

SEQ ID NO: 10 - protein, 15 aa, Artificial Sequence
  Synthetic construct
GCLASESSEGHVTGS

SEQ ID NO: 11 - protein, 15 aa, Artificial Sequence
  Synthetic construct
EAELQEKVSMCRSRS

SEQ ID NO: 12 - protein, 15 aa, Artificial Sequence
  Synthetic construct
HLDMLRHLYQGCQVV

SEQ ID NO: 13 - protein, 17 aa, Artificial Sequence
  Synthetic construct
RLRIVRGTQLFEDNYAL

SEQ ID NO: 14 - protein, 18 aa, Artificial Sequence
  Synthetic construct
TQRCEKCSKPCARVCYGL

SEQ ID NO: 15 - protein, 15 aa, Artificial Sequence
  Synthetic construct
GVGSPYVSRLLGICL

SEQ ID NO: 16 - protein, 15 aa, Artificial Sequence
  Synthetic construct
PAREIPDLLEKGERL

SEQ ID NO: 17 - protein, 15 aa, Artificial Sequence
  Synthetic construct
TLERPKTLSPGKNGV

SEQ ID NO: 18 - protein, 9 aa, Artificial Sequence
  Synthetic construct
KIFGSLAFL

SEQ ID NO: 19 - protein, 9 aa, Artificial Sequence
  Synthetic construct
RLLQETELV